(19) United States
(12) Patent Application Publication
(10) Pub. No.: US 2005/0177039 A1
(43) Pub. Date: Aug. 11, 2005

(54) Chronically implantable an artifact-free biomedical electrode assemblies

(76) Inventors: William J. Mills, Chino Hills, CA (US); Steven J. Pettinger, Wilmington, DE (US)

Correspondence Address: CHRISTIE, PARKER & HALE, LLP, PO BOX 7068, PASADENA, CA, 91109-7068, US

(21) Appl. No.: 10/981,310

(22) Filed: Nov. 3, 2004

Related U.S. Application Data
(60) Provisional application No. 60/516,694, filed on Nov. 3, 2003.

(51) Int. Cl. A61B 5/04

(52) U.S. Cl. 600/373; 600/378

(57) ABSTRACT

Several embodiments of body-compatible biomedical electrodes suitable for long-term implantation, and substantially free of detection of unwanted artifact signals. The electrodes are useful both for sensing body potentials, and for delivery of stimulating electrical signals. The electrodes can be coupled to implanted telemetry circuits, or connected to external electronic devices.

### CROSS-REFERENCE TO RELATED APPLICATION

This application claims the priority benefit of U.S. Provisional Application 60/516,694 filed Nov. 3, 2003, the disclosure of which is incorporated herein by reference.

### BACKGROUND OF THE INVENTION

There exists a need for biomedical electrodes which are suitable for long-term implantation, and for either stimulation of brain, cardiac or muscle function, or for signal sensing to enable monitoring or recording of neurological electrical signals and the like. For example, monitoring of brain electrical activity (electroencephalogram or “EEG”), or muscles (electromyogram or “EMG,” and electrocardiogram or “ECG”). An immediate application relates to such studies in animal experimentation.

The several electrode configurations of this invention are significant improvements with respect both to materials chosen for long-term implantation without tissue erosion, inflammation, or infection, and to elimination of spurious electrical signals by isolation of the electrode from interfering biopotential signals. The electrodes are bidirectional in that they are useful for either sensing biopotentials, or for delivering stimulating signals. The electrodes can be used with implanted electronics and telemetry transmitters, or by connection (through a transcutaneous skin exit) to external signal-conditioning and recording equipment.

### SUMMARY OF THE INVENTION

A chronically implantable biomedical electrode assembly, useful for delivering stimulating electrical signals, or for detecting tissue or muscle potentials. The assembly is constructed of body-compatible materials, and is substantially free of detection of unwanted artifact signals.

### BRIEF DESCRIPTION OF THE DRAWINGS

FIG. 1 is a side sectional view of a first electrode assembly for monitoring in contact with dura mater tissue of the brain;

FIG. 2 is a top view of the first assembly;

FIG. 3 is a bottom view of the first assembly;

FIG. 4 is a side sectional elevation of a second electrode assembly for deep-brain positioning of the electrode;

FIG. 5 is a sectional side elevation of a third electrode assembly similar to the second assembly, but having multiple electrodes, and an optional flexible electrode-supporting shaft;

FIG. 6 is a partial sectional elevation of a skull and brain with implanted first and third electrode assemblies;

FIG. 7 is a side sectional elevation of a fourth electrode assembly for muscle implantation;

FIG. 8 is a bottom view of the fourth assembly;

FIG. 9 is a side sectional elevation of a fifth electrode assembly similar to the fifth assembly, but having multiple electrodes;

FIG. 10 is a bottom view of the fifth assembly; and

FIG. 11 is a side sectional view of the fifth electrode assembly as implanted between two muscle layers.

### DESCRIPTION OF THE PREFERRED EMBODIMENTS

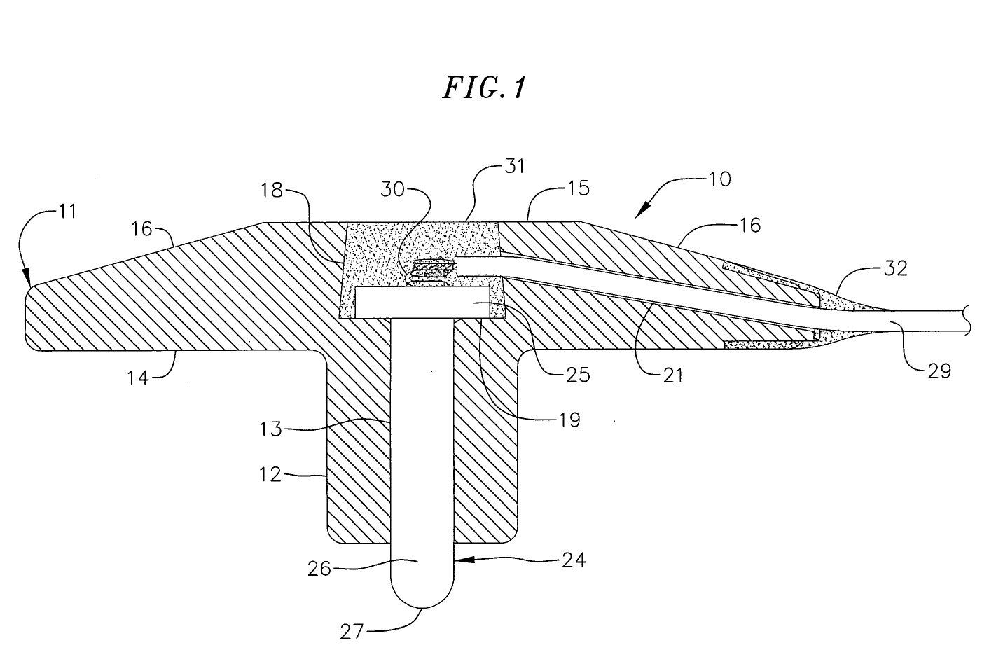

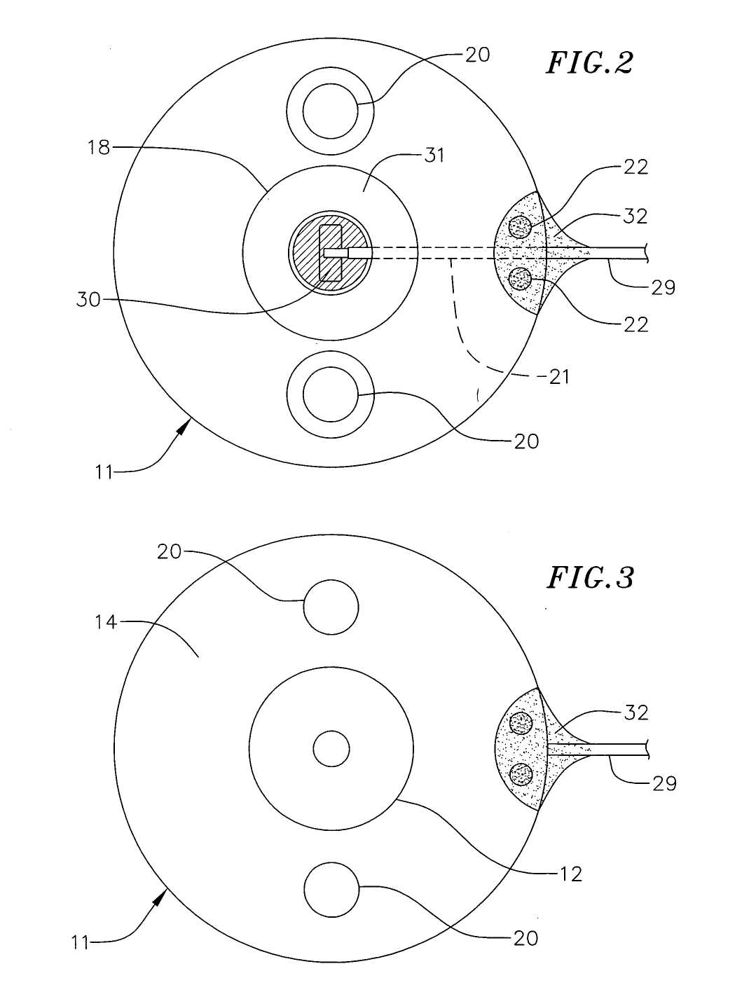

A first electrode assembly 10 is shown in FIGS. 1-3, and is especially suitable for implantation beneath the skull for sensing electrical EEG activity in a specific area of the brain's dura mater, or for delivering electrical signals to such area. The assembly has a circular button-like base 11 with a downwardly extending central tubular section 12 with a central opening 13. An undersurface 14 of the base is flat, and the base upper surface has a central flat section 15, and a downwardly tapered side section 16. A central opening 18 extends downwardly, and is tapered outwardly to a flat bottom surface 19 surrounding an upper end of opening 13.

A pair of recessed bone-screw openings 20 extend through the base, and are spaced apart 180 degrees on opposite sides of central opening 18. A tubular opening 21 extends from a side edge of the base into central opening 18, and a pair of circular passages 22 are formed through the base on opposite sides of and adjacent the outer end of tubular opening 21. Base 11 is made of a nonconductive tissue-compatible rigid plastic such as an acetal-resin polymer marketed under the trademark Delrin®.

A conductive electrode 24 has an enlarged circular head 25, with a downwardly extending pin 26 making a press fit in base central opening 13. A rounded lower end 27 of the pin extends beneath the lower end of tubular section 12. The electrode is made of a conductive and tissue-compatible nontarnishing metal such as type Ti6A14V titanium.

A lead wire 29 with biocompatible shielding, and for either external connection, or to connection with implanted circuitry, is fitted into tubular opening 21 to extend into central opening 18. A short folded section of annealed nickel ribbon 30 is welded to the top of electrode head 25, and soldered to a stripped inner end of the lead wire. Opening 18 is then filled with an epoxy material 31 (type 6203FF is suitable) to be level with flat upper surface 15 of the base. The junction of the lead wire at the inlet of opening 21 is stabilized and sealed with a layer of RTV sealant 32 (available from Dow Corning) applied over slight recesses of the upper and lower edges of side section 16 adjacent the inlet. The RTV sealant penetrates and fills passages 22 to form a secure bond.

Assembly 10 is compact, and base 11 typically has an outside diameter of about one-half inch, and electrode 24 has an overall axial length of about 0.2 inch. The diameter of electrode pin 26 is about 0.04 inch. Lead wire 29 preferably uses a stainless-steel conductor, and biocompatible insulated wires of this type are available from Cooner Wire in Chatsworth, Calif.

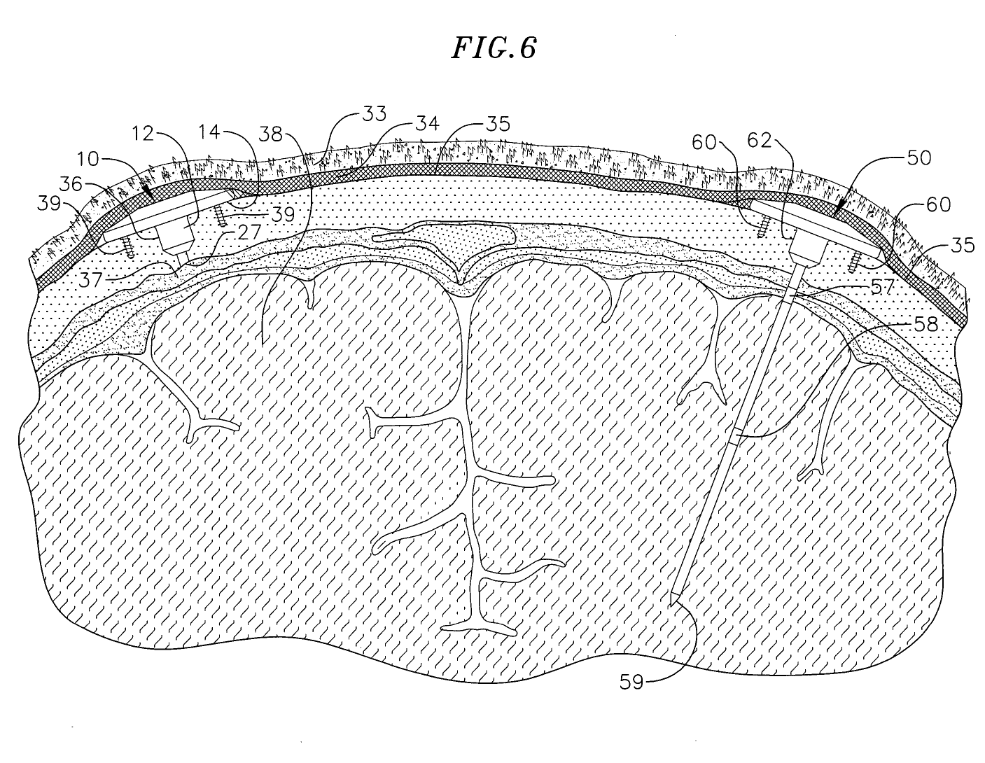

FIG. 6 illustrates implanted electrode assembly 10 as positioned beneath scalp 33 and underlying muscle layer 34, and with underside 14 of base 11 fitted against skull 35. Tubular section 12 is fitted into a drilled passage 36 through the skull to place electrode lower end 27 against dura mater 37 of brain 38. The electrode assembly is secured against the skull by a pair of self-tapping titanium (type TiGAL7Nb is suitable) bone screws 39.

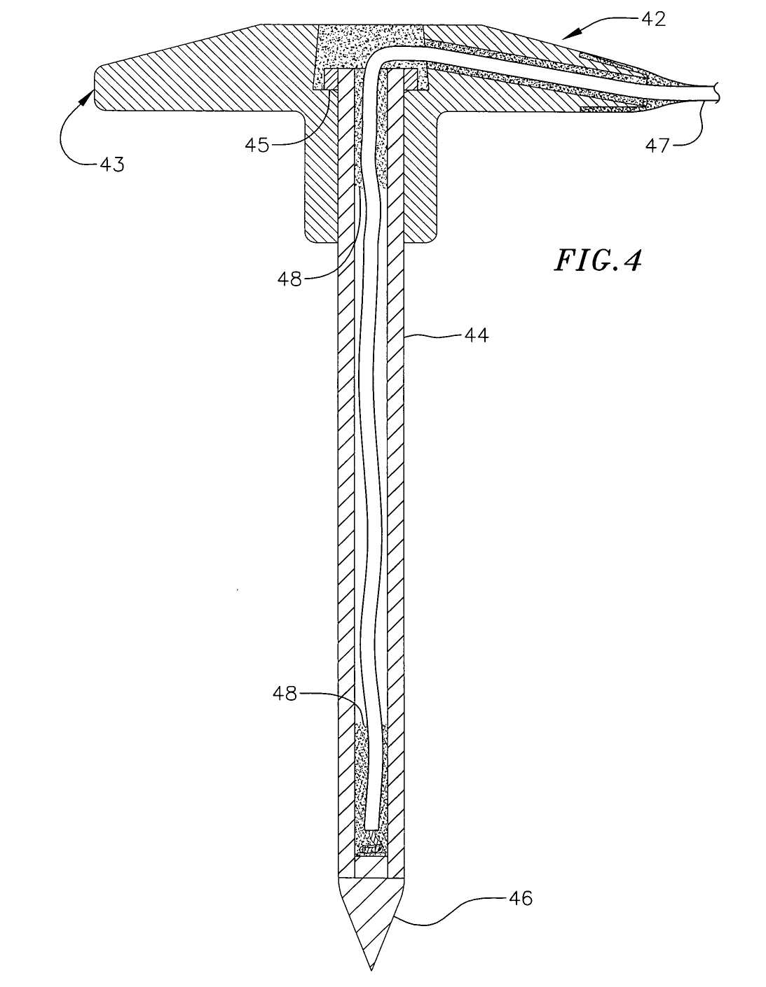

FIG. 4 shows a second embodiment of an electrode assembly 42 for deep-brain implantation. Assembly 42 has a base 43 which corresponds to base 11 described above. An elongated rigid plastic tube 44 is fitted into the base central tubular section, and has a plastic collar 45 (Delrino plastic is again suitable) secured at its upper end, the collar resting on the flat bottom surface of the base upper-central opening. Tube 44 is made of a biocompatible material such as polysulfone, polyetheretherketone, or Delrin® plastic.

A tapered titanium electrode 46 is press fitted into the bottom of tube 44, and is secured (by a welded and soldered nickel ribbon as described above) to the stripped end of a shielded lead wire 47 extending through base 43 and downwardly through tube 44. The upper and lower ends of the tube are filled with RTV sealant 48.

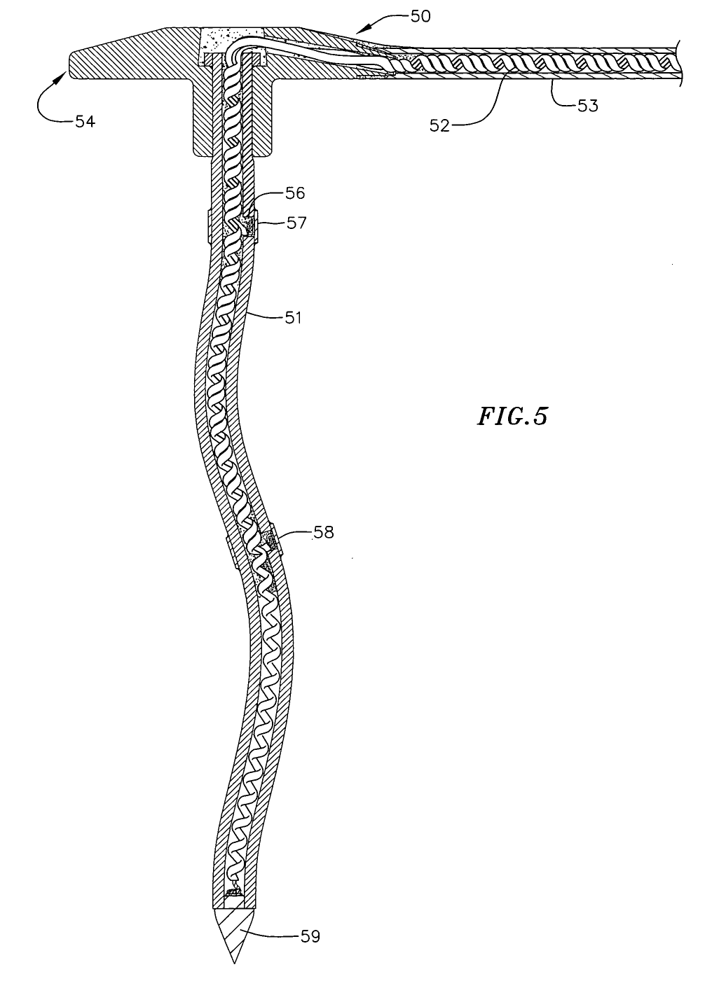

FIG. 5 shows a third electrode assembly 50 which is similar to assembly 42, but features multiple electrodes, and an optional flexible plastic tube 51 which may be favored for certain types of deep-brain implantations. Three coiled and shielded independent lead wires 52 surrounded by silicone tubing 53 are fed through a base 54 (corresponding to bases 11 and 43 as described above), and to extend downwardly into tube 51.

One of the lead wires is stripped, and welded/soldered as already described through an opening 56 in the sidewall of tube 51 to a titanium ring electrode 57 press fitted over the tube. A second lead wire is similarly secured to a second titanium ring electrode 58 spaced further down the tube. A third lead wire extends to the bottom of the tube for welded/soldered attachment to a tapered titanium tip electrode 59 corresponding to electrode 46 of assembly 42. The tube interior spaces adjacent the lead wire and electrode interfaces are again filled with an RTV sealant.

Referring again to FIG. 6, electrode assembly 50 (this time with a straight and rigid plastic tube supporting the electrodes) is secured at its base to skull 35 by a pair of titanium bone screws 60. The base tubular section and electrode-supporting tube extend through a drilled skull passage 61 to position the ring and tip electrodes at various levels of the brain.

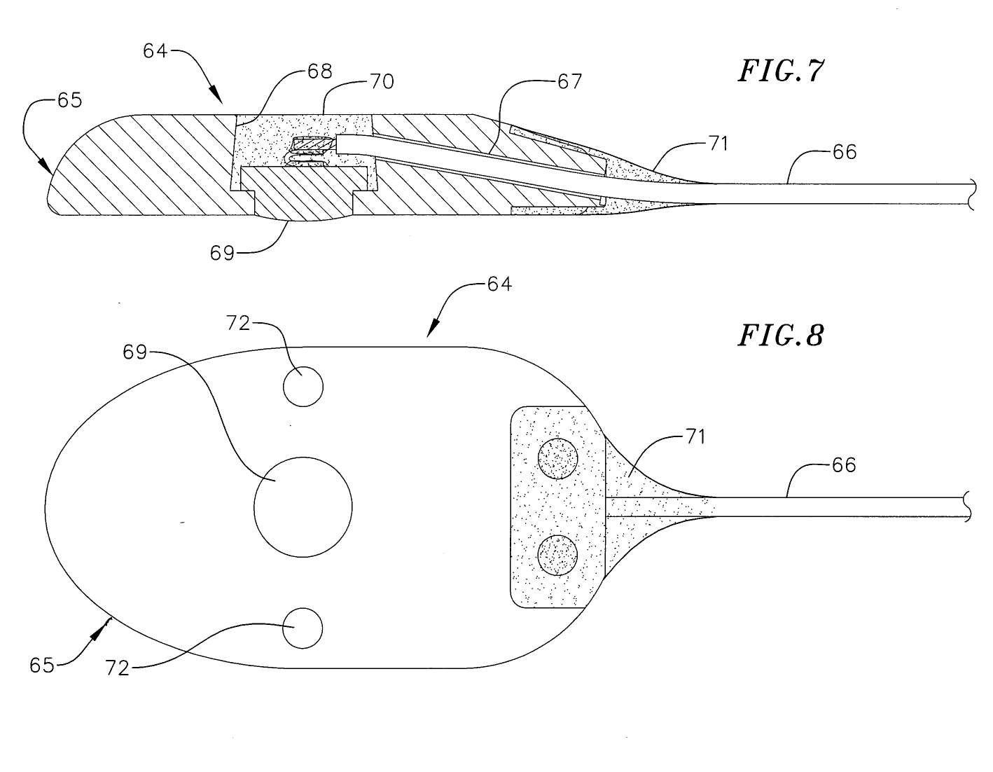

FIGS. 7 and 8 show a fourth electrode assembly 64 for muscle stimulation, or to detect electromyogram signals. The assembly has a base 65 similar to those described above, but having an oval shape in plan view (FIG. 8). A shielded lead wire 66 extends through a tubular passage 67 in the base to a base upper-central opening 68. A titanium electrode 59 is seated in opening 68, and a rounded electrode tip extends slightly below the undersurface of the base. A stripped inner end of the lead wire is soldered to a nickel ribbon which is welded to the electrode head as already described. The upper part of opening 68 is filled above the electrode head with epoxy material 70, and the lead wire is paired into the housing by RTV sealant 71, again as described above. A pair of holes 72 through the base on opposite sides of the electrode are provided to enable sutured attachment of the assembly to muscle.

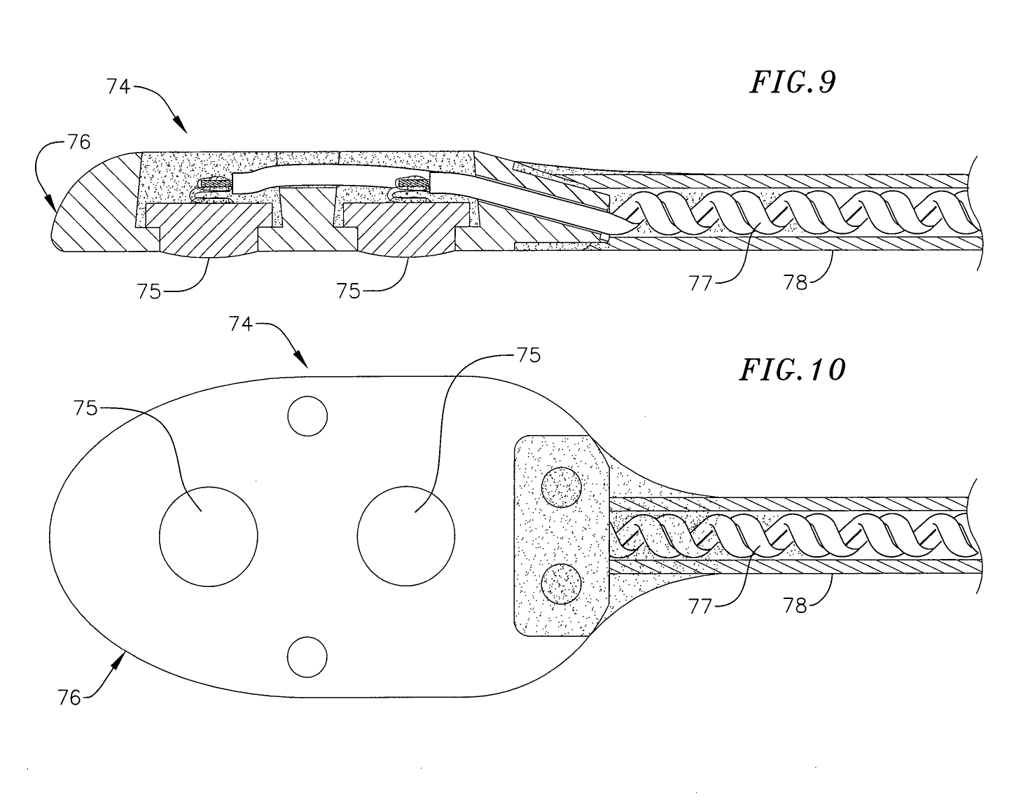

FIGS. 9 and 10 show a fifth electrode assembly 74 which is similar to assembly 64, but which accommodates two spaced-apart titanium electrodes 75 mounted in a base 76. Two coiled lead wires 77 extend through a silicone tube 78 for attachment to the electrode heads as already described.

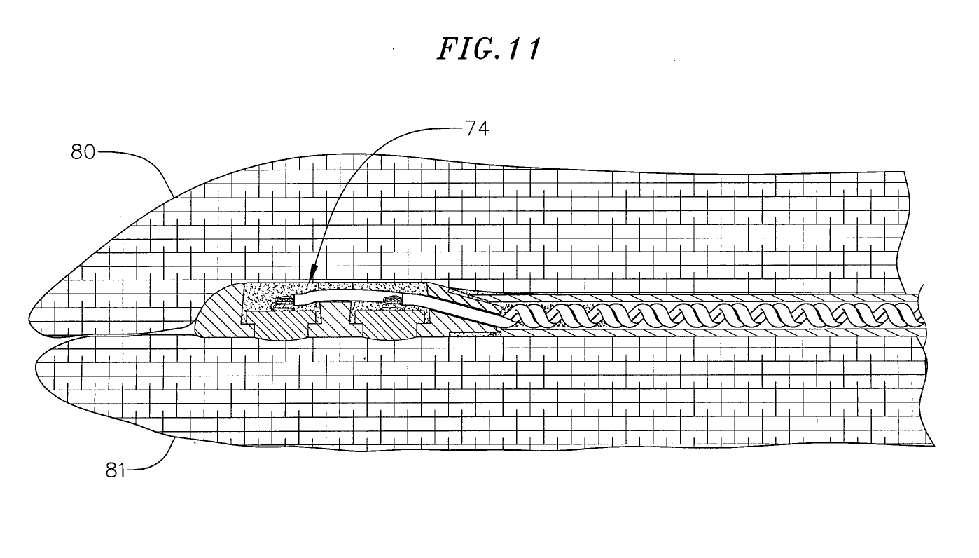

FIG. 11 shows electrode assembly 74 as implanted between upper and lower muscle layers 80 and 81. The dual electrodes are in contact with the lower muscle layer, and electrically isolated from the upper muscle layer. Again, these electrode assemblies are bidirectional, and can be used for sensing muscle potentials, or for delivery of stimulating signals.

There have been described several embodiments of bidirectional medical electrode assemblies made of materials which are body compatible, and suitable for long-term implantation without adverse tissue reaction. The electrodes are “site specific” in that they are isolated from and insensitive to adjacent non-target tissue potentials. As compared to prior-art conductor wires secured to bone screws, and fine wire electrodes implanted in the brain, the electrodes of this invention are substantially free of signal attenuation, interference or cross talk from overlying muscles, and noise and induced lead-whips potentials.

1. A chronically implantable biomedical electrode assembly having a base with a central downwardly extending hollow section, a conductive electrode seated in the base, and extending through and beyond the hollow section, and a lead wire through an opening in the base into electrical connection with an upper end of the electrode, all components being body compatible without causing adverse tissue reaction.

2. The electrode assembly defined in claim 2, wherein the electrode is sensitive only to body material contacted by an extending tip of the electrode, thereby avoiding sensing of interfering biopotential signals.

3. The electrode assembly defined in claim 1, wherein the electrode is elongated for deep-brain penetration, and surrounded by a tube secured to the base.

4. The electrode assembly defined in claim 3, wherein the lead wire has multiple independent conductors, and a plurality of tissue contacting electrodes connected to the conductors.

5. The electrode assembly of claim 1 as configured for implantation between muscle layers, the electrode having a conductive tip which extends only slightly from the hollow section.

6. The electrode assembly of claim 1, wherein the base is made of an acetal-resin polymer, and the electrode is titanium.
